(19) United States
(12) Patent Application Publication
(10) Pub. No.: US 2011/0014638 A1
(43) Pub. Date: Jan. 20, 2011

(54) SOLUBLE FAS IN ACUTE CORONARY SYNDROMES DIAGNOSIS

(75) Inventors: François Madore, Outremont (CA); Marie-Josée Hebert, Outremont (CA); Héloise Cardinal, Montreal (CA); Peter Bogaty, Hampstead (CA); James Brophy, Westmount (CA)

Correspondence Address: LANDO & ANASTASI, LLP, ONE MAIN STREET, SUITE 1100, CAMBRIDGE, MA, 02142, US

(73) Assignees: Hopital Du Sacre-Coeur De Montreal, Montreal, QC (CA); Centre Hospitalier De L'Universite De Montreal, Montreal, QC (CA)

(21) Appl. No.: 12/740,902

(22) PCT Filed: Oct. 31, 2008
(86) PCT No.: PCT/CA08/01921

§ 371 Date: Sep. 29, 2010

Related U.S. Application Data
(60) Provisional application No. 60/983,979, filed on Oct. 31, 2007.

(51) Int. Cl. G01N 33/53

(52) U.S. Cl. 435/7.92

(57) ABSTRACT

The present technology provides methods and materials for the early clinical detection of acute coronary syndromes in a subject. The methods and materials comprise measuring the amount of soluble Fas in a sample obtained from a subject, comparing the amount of soluble sFas with a reference value and detecting the presence of acute coronary syndromes.

### FIELD OF TECHNOLOGY

The present technology relates to the identification and use of soluble (sFas) as a diagnostic marker for acute coronary syndromes (ACS). In various aspects, the present technology relates to methods, assays and materials for the early diagnosis of ACS in a subject by determining the amount of sFas in a sample obtained from the subject.

### BACKGROUND OF THE INVENTION

Acute coronary syndromes (ACS) represent an important medical and economic burden in North America, causing significant morbidity and mortality'. The diagnosis and early management of ACS remain challenging as the clinical presentation may be atypical, the electrocardiogram (ECG) non-diagnostic, and specific necrosis markers (such as creatine kinase MB (CK-MB) and troponin) are often negative at the time of patient admission². Delays in diagnosis can lead to less aggressive early management strategies in patients who suffer from ACS, and unwarranted observation periods and resource deployment in the majority of patients with chest pain who ultimately are found not to have ACS.

ACS result from the sudden total or sub-total occlusion of coronary vessels when an inflammatory atheromatous plaque ruptures and exposes its pro-coagulant material to circulating blood, promoting clot formation and distal cardiac muscle ischemia/necrosis³. Recent evidence suggests that apoptosis of vascular smooth muscle cells (VSMC) and macrophages are involved in plaque instability and rupture^{4;5}. Furthermore, the pro-coagulant influence of dysfunctional and apoptotic endothelial cells (ECs)⁶, as well as increased levels of circulating apoptotic cell remnants⁷, are believed to increase thrombogenicity. Hence, detection of soluble markers indicative of increased apoptosis may allow for an earlier and improved diagnostic accuracy of ACS.

Fas is a 45 kDa transmembrane protein that initiates an apoptotic signal in a wide variety of cell types when bound to its natural ligand⁸.

In small studies, high levels of Fas's soluble form, sFas, have been reported in patients with unstable angina (UA)⁹ and myocardial infarction (MI) ¹⁰. However, these studies did not adjust for potential confounders, did not test the diagnostic utility of these markers, and did not assess whether the levels of sFas rose earlier than CK-MB and troponin.

The clinical utility of sFas as a diagnostic marker has never been explored previously. Control groups in prior studies have always included patients with stable angina²¹ or normal controls^{9;10;21} , making it difficult or impossible to evaluate whether measurement of sFas might improve diagnostic accuracy in a context where ACS is suspected. Prior studies have not characterized the kinetics of sFas expression following an ACS episode. Furthermore, no methods for measuring sFas have been validated for clinical use.

Therefore, there remains a need in the art for identification of a biological marker the level of which in a patient rises faster than the markers currently used and which improves diagnostic accuracy of the diagnosis of ACS.

### SUMMARY OF THE INVENTION

In one aspect, the present invention provides a method for early diagnosis of the presence of acute coronary syndromes (ACS) in a subject, comprising the steps of: (a) determining the amount of soluble Fas (sFas) or a fragment thereof in a sample obtained from the subject; and (b) comparing the amount of sFas or the fragment thereof in the sample with a reference value, wherein the amount of sFas or the fragment thereof in the sample as compared with the reference value indicates the presence or absence of ACS.

In another aspect, the present invention provides a method for early diagnosis of the presence of acute coronary syndromes (ACS) in a subject, comprising the steps of: (a) determining the amount of soluble Fas (sFas) or a fragment thereof in a sample obtained from the subject; and (b) correlating the amount of sFas or the fragment thereof to the presence or absence of ACS in the subject by comparing the amount of sFas or the fragment thereof to a reference value, wherein when the amount of sFas or the fragment thereof exceeds the reference value, ACS is diagnosed in the subject.

In another aspect, the present invention provides a method for early diagnosis of the presence of acute coronary syndromes (ACS) in a subject, comprising the steps of: (a) determining the amount of soluble Fas (sFas) or a fragment thereof in a sample obtained from a subject; (b) determining the amount of at least one ACS marker in the sample; and (c) correlating the amounts obtained in (a) and (b) to the presence or absence of ACS in the subject by comparing the amount obtained in (a) to a reference value for sFas and comparing the amount obtained in (b) to a reference value for the at least ACS marker, wherein when at least the amount obtained in (a) exceeds the reference value for sFas, ACS is diagnosed in the subject.

In another aspect, the present invention provides a kit for early clinical diagnosis of the presence of acute coronary syndromes (ACS) in a subject, comprising: reagents for measuring the amount of soluble Fas (sFas) or a fragment thereof in a biological sample obtained from the subject; and instructions for use of the kit.

In another aspect, the present invention provides a kit for early clinical diagnosis of the presence of acute coronary syndromes (ACS) in a subject, comprising: a sFas binding agent, at least one detection reagent that indicates the amount of sFas or a fragment thereof present in a biological sample obtained from the subject; and instructions for use of the kit.

In another aspect, the present invention provides a kit for early clinical diagnosis of the presence of acute coronary syndromes (ACS) in a subject, comprising: a sFas binding agent, at least one sFas detection reagent that indicates the amount of sFas or a fragment thereof present in a sample, at least one ACS marker binding agent, at least one ACS marker detection reagent that indicates the amount of the ACS marker in a sample, and instructions to use the kit

In another aspect, the present invention provides use of soluble sFas as a marker for detection of acute coronary syndromes (ACS).

### BRIEF DESCRIPTION OF THE FIGURES

FIG. 1: is a graph showing the area under the Receiver Operating Curve for sFas alone.

FIG. 2: is a graph showing the area under the Receiver Operating Curve for multivariate model without sFas.

FIG. 3: is a graph showing the area under the Receiver Operating Curve for multivariate model with sFas.

### DETAILED DESCRIPTION

The implementations defined below are not intended to be exhaustive or to limit the technology to the precise forms disclosed in the following detailed description. Rather, the implementations are chosen and described so that others skilled in the art may appreciate and understand the general principles and general practices of the present technology.

Unless defined otherwise, all technical and scientific terms used herein have the same meaning as commonly understood by one of skill in the art to which the technology pertains.

For ease of reference, the following abbreviations and designations are used throughout:

ACS: acute coronary syndromes

sFas: soluble FAS

Fas-L: Fas ligand

MI: myocardial infarction

UA: unstable angina

ECG: electrocardiogram

CK: creatine kinase

CK-MB: creatine kinase from muscle and brain

ECs: endothelial cells

VSMC: vascular smooth muscle cells

ROC: receiver operating curve

ELISA: enzyme-linked immunosorbent assay

The present technology stems from, but is not limited to, the discovery that an increase in the amount of circulating apoptosis marker correlates with the occurrence of an ACS episode.

In various aspects, the technology defined herein relates to methods, assays and materials for detecting markers that are associated with the diagnosis of ACS in a subject, as well as using these markers in such diagnosis. The present study provides evidence that sFas is a reliable marker for early diagnosis of ACS.

The methods of the present technology include assaying a sample obtained from the subject for sFas, wherein the presence of an elevated level of sFas indicates the presence of ACS. This method can be performed in conjunction with one or more other tests, physical examination, and/or the taking of a medical history to allow a differential diagnosis of, e.g., myocarditis, ischemic heart disease, or dilated, hypertrophic, or restrictive cardiomyopathy. The tests and parameters employed in diagnosing these disorders are well known to those of skill in the art. These methods can be carried out on samples from asymptomatic subjects or subjects having one or more risk factors associated with, or symptoms of, ACS.

Among patients who present to an emergency department with chest pain believed to be of cardiac origin, the present studies demonstrate that plasma sFas levels are strongly associated with a final diagnosis of ACS, even after thorough adjustment for potential confounding factors and the case-definition biomarker variables CK and cardiac troponin. The measurement of sFas therefore adds incremental value for the diagnosis of ACS in this cohort.

In patients who had negative necrosis markers and no ischemic ECG changes, sFas remained strongly associated with a discharge diagnosis of ACS. sFas levels increased earlier than the diagnostic necrosis markers in patients who had ACS.

Fas is a transmembrane protein that initiates an apoptotic signal when bound to its ligand (Fas-ligand (Fas-L))⁸. Expression of Fas has been identified at sites of atherosclerotic lesions in both animal models and humans ¹³⁻¹⁵

Three cell lines undergo apoptosis in unstable plaques: VSMCs, macrophages and ECs. VSMCs express Fas at their surface^{16,} and high levels of Fas-L have been shown in inflammatory regions of human carotid plaques¹⁷. Lipid-filled macrophages also express Fas in atheromatous plaques, making it likely that these cells undergo apoptosis through this pathway¹³. ECs express little Fas at their surface under normal conditions. Nonetheless, in vitro data shows that pro-atherogenic factors such as oxLDLs¹⁸ induce apoptosis in ECs through the Fas pathway.

Coronary artery segments obtained from autopsies or atherectomies have revealed increased expression of pro-apoptotic genes¹⁹ and apoptotic VSMCs and macrophages⁵ in unstable plaques compared with stable plaques. Hence, an early increase in apoptosis markers during ACS episodes is biologically plausible.

The data presented herein show that sFas rises precociously in ischemia. Indeed, early (≦6 hours after the beginning of symptoms) versus late (>18 hours) measurements were similar (5.98 vs. 5.89 In pg/ml, p=0.37). In comparison, CK and troponin increased with time since the onset of symptoms.

The diagnosis of ACS remains challenging, especially in the frequent circumstances when chest pain is atypical and ECG changes are absent or non-diagnostic. In these cases, diagnosis is often delayed pending an increment in myocardial necrosis markers, since 40-60% of patients present with initially non-diagnostic concentrations²⁰. In other cases, the diagnosis remains uncertain.

The data presented herein demonstrate, inter alia, the existence of a reliable an accurate clinical correlation between the precocious rise in sFas and the presence of ACS. The present technology provides a method for early diagnosis of ACS through measurement of sFas levels.

As used herein and the appended claims, the singular forms “a”, “an” and “the” include plural referents unless the context clearly dictates otherwise. Thus, it is intended that the present technology covers such modifications and variations as come within the scope of the appended claims and their equivalents. As used herein, and in the claims, the term “comprising” is inclusive or open-ended and does not exclude additional unrecited elements, compositional components, or method steps. Accordingly, such term is intended to be synonymous with the words “has”, “have”, “having”, “includes”, “including”, and any derivatives of these words.

As used herein, the term soluble Fas (sFas) encompasses sFas protein and fragments thereof, SFAS receptor (sFasR) and fragments thereof. Other acronyms that may be used to designate sFas include ALPS1A, APO-1, APT1, CD95, FAS1, FASTM and TNFRSF6.

By “peptide”, “polypeptide” or “protein” is meant any chain of amino acids, regardless of length or post-translational modification (e.g., glycosylation or phosphorylation), or chemical modification, or those containing unnatural or unusual amino acids such as D-Tyr, ornithine, amino-adipic acid. The terms are used interchangeably in the present application. Proteins or polypeptides defined herein are contemplated to include any fragments, any analogs, any variants or homologs thereof that, in particular, preserve the activity of the full-length peptide and/or are immunologically detectable.

The term “fragments” refers to amino acid fragments of a peptide such as, for example, sFas. It will be understood that fragments of a peptide are shorter than the full-length peptide. For example, fragments of sFas that preserve the biological activity of the peptide and/or fragments that are immunologically detectable are encompassed by the present technology.

The proteins and peptides defined herein may also be part of a fusion protein. It is often advantageous to include an additional amino acid sequence which contains, for example, secretory or leader sequences, pro-sequences, sequences which aid in purification such as multiple histidine residues, FLAG epitope tags or similar metal affinity tags, or an additional sequence for stability during recombinant protein production.

Functional and structural analogs of a peptide or fragments thereof are also encompassed by the present technology. General methods and synthetic strategies used in providing functional and structural analogs of a peptide or fragments thereof are commonly used and well known in the art and are described in publications such as “Peptide synthesis protocols” ed, M. W. Pennigton & B. M. Dunn. Methods in Molecular Biology. Vol 35. Humana Press, NJ., 1994.

The term “homology” refers to sequence similarity between two peptides while retaining an equivalent biological activity. Homology can be determined by comparing each position in the aligned sequences. A degree of homology between amino acid sequences is a function of the number of identical or matching amino acids at positions shared by the sequences so that a “homologous sequence” refers to a sequence sharing homology and an equivalent function or biological activity. Assessment of percent homology is known by those of skill in the art.

Methods to determine identity and similarity of peptides are codified in publicly available computer programs. Preferred computer program methods to determine identity and similarity between two sequences include, but are not limited to, the GCG program package, BLASTP, BLASTN, and FASTA. The BLAST X program is publicly available from NCBI and other sources. The well known Smith Waterman algorithm may also be used to determine identity. Preferred parameters for polypeptide sequence comparison include the following:

Algorithm: Needleman and Wunsch, J. MoI Biol. 48: 443-453 (1970);

Comparison matrix: BLOSSUM62 from Hentikoff and Hentikoff, Proc. Natl. Acad. Sci. USA. 89:10915-10919 (1992);

Gap Penalty: 12; Gap Length Penalty: 4.

A program useful with these parameters is publicly available as the “gap” program from Genetics Computer Group, Madison, Wis. The aforementioned parameters are the default parameters for amino acid sequence comparisons (along with no penalty for end gaps).

In specific, but non-limiting, implementations, the polypeptides are used in a form that is “purified”, “isolated” or “substantially pure”. The polypeptides are “purified”, “isolated” or “substantially pure” when they are separated from the components that naturally accompany them. Typically, a compound is substantially pure when it is at least 60%, 65%, 70%, 75%, 80%, 85%, 90%, 95%, 96%, 97%, 98%, or 99%, by weight, of the total material in a sample.

The polypeptides defined herein may be prepared in any suitable manner as known in the art. Such polypeptides include isolated naturally occurring polypeptides, recombinantly produced polypeptides, synthetically produced polypeptides, or polypeptides produced by a combination of these methods. Means and methods for preparing such polypeptides are well known in the art.

The term “antigen” here refers to molecules (from whatever source, natural or man-made) which induce an immune reaction when recognized by the host's immune system.

The term “antibody” refers to a class of serum proteins which specifically bind to an antigen which induced the formation of the antibody.

The term “epitope” as used here refers to a molecular region on the surface of an antigen that is capable of specific binding to an antibody against the antigen. The epitope may be linear, i.e. a fragment of contiguous amino acids from the sequence of the antigen, or it may be formed from different regions of the linear sequence.

Certain aspects of the present technology use polynucleotides. These include isolated polynucleotides which encode for example the polypeptides, fragments and analogs defined in the application. As used herein, the term “polynucleotide” refers to a molecule comprised of a plurality of deoxyribonucleotides or nucleoside subunits. The linkage between the nucleoside subunits can be provided by phosphates, phosphonates, phosphoramidates, phosphorothioates, or the like, or by non-phosphate groups as are known in the art, such as peptoid-type linkages utilized in peptide nucleic acids (PNAs). The linking groups can be chiral or achiral. The oligonucleotides or polynucleotides can range in length from 2 nucleoside subunits to hundreds or thousands of nucleoside subunits. While oligonucleotides are preferably 5 to 100 subunits in length, and more preferably, 5 to 60 subunits in length, the length of polynucleotides can be much greater (e.g., up to 100). The polynucleotide may be any of DNA and RNA. The DNA may be in any form of genomic DNA, a genomic DNA library, cDNA derived from a cell or tissue, and synthetic DNA. Moreover, the present invention may, in certain aspects, use vectors which include bacteriophage, plasmid, cosmid, or phagemid.

The term “detecting” may be used herein in the broadest sense to include both qualitative and quantitative measurements of a target molecule or analyte. Qualitatively, the detecting methods as described herein are used to identify the mere presence of a molecule of interest in a biological sample. The method may be used to test whether the molecule of interest in a sample is present at a detectable level. Quantitatively, the method measures the amount, the levels or the concentration of the molecule of interest in a sample and further compares the amount, levels or concentration of the molecule to a reference value. In a further sense, the term detecting may also refer to detection or recognition of a disease or condition in a subject.

The term “analyte” as used herein, refers to the substance to be detected, which may or may not be present in the sample. The analyte can be any substance for which there exists a naturally-occurring or a man-made specific binding partner. Thus, an analyte is a substance that can bind to one or more specific binding partners in an assay.

The term “diagnosis” or “diagnosing” is used herein to refer to the detection or recognition of a disease or condition by its outward signs and symptoms. The term “diagnosis” or “diagnosing” may also encompass the detection or recognition of the presence of ACS.

The methods and materials encompassed by the present technology are also useful for aiding detection and/or recognition and/or diagnosis of ACS.

The methods and materials encompassed by the present technology are also useful for the detection and/or recognition and/or the diagnosis of MI as well as for aiding detection and/or recognition and/or diagnosis of MI. The methods and materials encompassed by the present technology are also useful for the detection and/or recognition and/or the diagnosis of UA as well as for aiding detection and/or recognition and/or diagnosis of UA.

The term “correlating” as used herein refers to the use of a diagnostic marker to compare the presence or amount of the marker in a subject to its presence or amount in subjects known to suffer from, or known to be at risk of, a given condition, or in subjects known to be free of a given condition, i.e. “normal subjects”. For example, a marker level in a subject sample can be compared to a level known to be associated with ACS. The sample's marker level is said to have been correlated with a diagnosis; that is, the skilled artisan can use the marker level to determine whether the patient suffers from ACS, and respond accordingly. Alternatively, the sample's marker level can be compared to a marker level known to be associated with a good outcome (e.g., the absence of ACS), such as an average level found in a population of normal individuals.

The expression acute coronary syndromes (ACS) as used herein refers to a set of signs and symptoms (syndrome) related to the heart. ACS is compatible with a diagnosis of acute myocardial ischemia, but it is not pathognomonic. The subtypes of acute coronary syndromes include unstable angina and myocardial infarction, in which heart muscle is damaged. These types are named according to the appearance of the electrocardiogram (ECG/EKG) as non-ST segment elevation myocardial infarction (NSTEMI) and ST segment elevation myocardial infarction (STEMI). A person skilled in the art would appreciate that there can be some variations as to which forms of symptoms related to the heart that are classified under ACS.

The terms “ischemia” or “ischemic” relates to damage to the myocardium as a result of a reduction of blood flow to the heart. The term “unstable angina” generally relates to myocardial ischemia. A person skilled in the art will be familiar with these terms. These terms are described at least in “The Merck Manual of Diagnosis and Therapy” Seventeenth Edition, 1999, Ed. Keryn A. G. Lane, pp. 1662-1668. The term ischemia is also related to what one skilled in the art would consider as minor myocardial injury or damage. The term ischemia is described at least in the Journal of the American College of Cardiology 36, 959-969 (2000).

The term “necrosis” relates to myocardial cell death as a result of a reduction or stoppage of blood flow to the heart. The term “myocardial infarction” generally relates to myocardial necrosis. One skilled in the art will recognize these terms, which are described at least in “The Merck Manual of Diagnosis and Therapy” Seventeenth Edition, 1999, Ed. Keryn A. G. Lane, pp. 1668-1677. The term necrosis is also related to what one skilled in the art would consider as major myocardial injury or damage. The terms myocardial infarction and necrosis are described at least in the Journal of the American College of Cardiology 36, 959-969 (2000).

The term “marker” as used herein refers to an indicator or a characteristic trait of a disease or condition that facilitates diagnosis.

In a specific, but non-limiting implementation, the present technology provides a method for diagnosing a subject for the presence of ACS. The method can be practiced using a sample that may contain sFas.

The term “sample” or “biological sample” refers to any biological substance that may contain the analyte or the marker of interest. A sample can be biological fluid, such as whole blood or whole blood components including red blood cells, white blood cells, platelets, plasma, serum, ascites, urine, vitreous fluid, lymph fluid, synovial fluid, follicular fluid, seminal fluid, amniotic fluid, milk, saliva, sputum, tears, perspiration, mucus, cerebrospinal fluid, and other constituents of the body.

The sample is a body sample from any animal, but preferably is from a mammal, more preferably from a human subject, most preferably from a subject susceptible of having ACS. Specifically such biological sample may be from clinical patients. The typical sample herein is blood, plasma, serum, tissue extracts, urine or any combination or any mixture thereof.

As used herein, the term “plasma” is intended to mean acellular fluid in which blood cells are suspended. Plasma samples may also be citrated.

As used herein, the term “subject suspected of having ACS” refers to a subject that presents one or more symptoms indicative of ACS episodes such as for example, atypic chest pain that may be experienced as tightness around the chest and radiating to the left arm and the left angle of the jaw, diaphoresis (sweating), nausea, vomiting, shortness of breath, palpitations, anxiety, a sense of impending doom and/or a feeling of being acutely ill. Generally, a subject suspected of having ACS has not been tested for ACS. However, a “subject suspected of having ACS” encompasses a subject who previously had one or more ACS episodes (e.g., an individual in remission) but is susceptible of having another episode or is having another episode.

Techniques useful for obtaining a sample, such as for example, a blood sample or a plasma sample, from a subject are well known in the art pertaining to this technology and will be recognized by skilled artisans with general knowledge of the art. Blood samples may be obtained initially using conventional techniques, such as, but not limited to, phlebotomy, with plasma formed according to well known techniques in the art.

It is understood that for use in the methods and materials defined herein, the samples may be treated or processed to remove particulate matter such as whole cells or cell debris so that the actual sample for use in the methods and materials are “cell-free” fluid samples. Procedures for removing cells and debris from biological fluids are known in the art. Such treated samples may give better results free of interference from the cells and debris. The samples may also be diluted in a suitable buffer solution or concentrated if necessary. A person skilled in the art will be familiar with buffer solutions that can be used.

In one specific, but non-limiting, implementation, methods are defined herein for detecting, quantifying or measuring the amount, level or concentration of sFas in a sample obtained from a subject, typically blood samples, most typically plasma samples, taken from a subject shortly after experiencing a possible ACS episode.

In another specific, but non-limiting, implementation, a diagnosis method for the presence of ACS in a subject includes the detection, quantification or measurement of the amount, level or concentration of Fas mRNA in a sample obtained from the subject, typically blood samples, most typically plasma samples, taken from a subject shortly after experiencing a possible ACS episode.

The precocious rise in sFas following an ACS episode (6 hours) permits an earlier diagnostic and provides more suitable guidance for choosing a treatment when compared to a method that measure the level of traditional ACS markers (such as, for example, troponin) which take up to 8, 12, 18 or 24 hours before rising, thereby delaying diagnosis and treatment accordingly. Therefore, preferably, the sample will be taken from the subject within 24 hours of the onset of the possible ACS episode, more preferably within 12 hours of the onset, and more preferably within 6 hours of the onset.

In a further specific, but non-limiting implementation, the method defined herein is used for early diagnosis of ACS. As used herein, the term “early” refers to a diagnosis that is obtained at an earlier time after the onset of possible ACS than a diagnosis that is obtained using conventional ACS markers such as, for example, CK and troponin. The advantages for an “earlier” diagnosis of ACS are defined herein and will be apparent to a person skilled in the art. For example, an early diagnosis of ACS is a diagnosis that is obtained 24 hours of the onset, preferably 12 hours of the onset, and most preferably, 6 hours of the onset of an ACS episode.

Measurement of sFas

In a specific, but non-limiting, implementation, the present technology provides methods for measuring the level of sFas in a sample obtained from a subject.

In a further specific, but non-limiting, implementation, the technology provides methods for measuring the level of sFas in a sample obtained from a subject which include contacting a sample obtained from a subject with a sFas binding agent. The method may also include additionally contacting the sample with one or more detection reagents which indicate the amount or concentration of sFas in the sample.

The amount, level or concentration of sFas may be measured using any suitable quantitative technique or semi-quantitative analytical technique. Such techniques include, but are not limited to, functional assays, enzymatic assays, enzyme-linked immunosorbent assays (ELISA), immunoassays, radioassays, chromatography assays, spectrophotometry assays, colorimetric assays, competitive binding assays and any combination thereof or any mixture thereof. A person skilled in the art would be familiar with these techniques as well as with other assays suitable for measuring the amount, level or concentration of sFas in a sample.

As used herein, the term “immunoassays” encompasses, but is not limited to, assay systems using techniques such as Western blots, radioimmuunoassays, ELISA, “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few.

As used herein, the expression “sFas binding agent” refers to any molecule or any organic compound or any inorganic compound naturally-occurring or man-made that selects for sFas protein, fragments thereof or derivatives thereof or that selects for Fas mRNA.

A fragment of sFas is a polypeptide having an amino acid sequence that is the same as part, but not all, of the sFas amino acid sequence. sFas fragments include, for example, truncation polypeptides having the amino acid sequence of sFas polypeptides, except for deletion of a continuous series of residues that includes the amino terminus, or a continuous series of residues that includes the carboxyl terminus or deletion of two continuous series of residues, one including the amino terminus and one including the carboxyl terminus. sFas fragments may also be characterized by structural or functional attributes such as, but not limited to, fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, transmembrane regions, and high antigenic index regions.

Examples of sFas binding agents include, but are not limited to, antibodies, antibody fragments and other equivalent binding substances which bind to an epitope within a region of sFas, sFas ligands and sFas substrates. In a specific, but non-limiting implementation, the sFas binding agent is an anti-sFas antibody. The anti-sFas antibody may be a polyclonal or a monoclonal antibody.

Other possible sFas binding agents include, but are not limited to, proteins, nucleic acids, polymers, carbohydrates, lipids, to name but a few. The sFas binding agents may further be labeled, probed or marked.

Antibodies against, for example, mouse or chicken Fas are commercially available from (Bender MedSystems, Campus Vienna Biocenter 2, A-1030, Vienna, Austria). Antibodies obtained from other species are also available elsewhere. A person skilled in the art will be familiar with commercial suppliers of anti-sFas antibodies.

The term “antibody fragment” refers to portions of an antibody molecule which retain the ability to bind to epitopes on the antigen. Such antibody fragments include the Fab, Fab', and F(ab')2 fragments of immunoglobulins.

An equivalent molecule of an antibody or an equivalent molecule of an antibody fragment refers to numerous recombinant molecules having the binding specificities of antibodies or of antibody fragments. Usually, an antibody molecule is prepared in a conventional manner, as commonly known in the art, and the gene encoding the antibody molecule isolated. After sequencing the antibody gene, recombinant nucleic acids can be isolated or synthesized and used to prepare binding proteins by a variety of known conventional methods.

Antibodies may be prepared by conventional techniques using antigenic and haptenic peptides which incorporate the epitope(s) of the conserved regions. The peptides may readily be prepared using known solid phase synthesis techniques and automated production equipment available from commercial vendors, such as Applied Biosystems, Foster City, Calif. The peptides so prepared may then be used as immunogens for producing polyclonal and/or monoclonal antibodies by well known techniques. The production of monoclonal antibodies is well described at least in literature; see, e.g., Harlow and Lane, eds., Antibodies: A Laboratory Manual, Cold Spring Harbor Laboratory, Cold Spring Harbor, N.Y.

In a further specific, but non-limiting implementation, the method for diagnosing ACS includes detecting and measuring the level of one or more additional ACS markers in a sample. In a specific example, the additional ACS marker is a necrosis factors. Additional ACS markers may include, but are not limited to, CK, CK-MB, troponin, myoglobin, myosin light chain, B-type natriuretic peptide (BNP), C-reactive protein (CRP), interleukins, TNF, adhesion molecules, aconitic acid, hypoxanthine, trimethylamine N-oxide, threonine, metabolites participating in the pyrimidine metabolism, in the tricarboxylic acid cycle and its upstream contributors, in the pentose phosphate pathway, markers of atherosclerotic plaque rupture, other markers that are presently under investigation. In a specific example, the troponins are troponin T and/or troponin I, and more preferably troponin T and troponin I are cardiac troponin T and/or cardiac troponin I. The measurement of these makers in a sample may be done concomitantly with the measurement of sFas, or may be done subsequently to the measurement of sFas. Other possible ACS markers may be obtained by examining the blood oxygen level, cardiac imaging, electrocardiography and the like.

The measurement of additional ACS markers may be carried out on the same sample used for measurement of sFas or on a different sample obtained from the same subject. If a different sample is to be used, it will be from the same type (e.g. plasma) and taken at approximately the same time as the sample obtained for measurement of sFas.

In a specific implementation, the measurement of additional ACS markers may be done as a confirmation measurement, i.e., confirming a diagnosis based on the measurement of sFas. The need and timing for performing such confirmatory measurement will be readily appreciated by those of skill in the art.

In a specific, but non-limiting implementation, the method may include quantifying one or more ACS markers in a sample by contacting the sample obtained from a subject with one or more ACS marker binding agents. As used herein, the expression “ACS marker binding agents” refers to any molecule or any organic compound or any inorganic compound naturally-occurring or man-made, that selects for an ACS maker protein fragments thereof or derivatives thereof, other than sFas. Specific examples of ACS marker binding agent include, but are not limited to, antibodies, antibody fragments and other equivalent binding substances which bind to an epitope within the region of other ACS markers such as those defined above. Other possible ACS marker binding agents include, but are not limited to, proteins, nucleic acids, polymers, carbohydrates, lipids, to name but a few. The ACS marker binding agents may further be labeled, probed or marked. The binding agents suitable for use in the method defined herein as well as the amount to use will be apparent to a person skilled in the art.

In a further implementation, the method includes quantifying one or more ACS markers in a sample by contacting the sample obtained from a subject with one or more ACS marker binding agents and with one or more detection reagents that indicate the amount of such ACS markers in a sample.

In a specific, but non-limiting implementation, the amount of sFas in a sample may be determined by contacting the sample with a sFas binding agent and measuring the amount of complex formed by interaction of sFas with the sFas binding agent. In this implementation, the sFas binding agent may be, for example, an anti-sFas antibody. The anti-sFas antibody interacts with sFas in the sample to form a complex antigen: antibody (sFas:anti-sFas antibody). The complex is washed from any unbound anti-sFas antibody and the amount of complex formed is measured using techniques that are well known in the art. In this method, the level of complex may be measured by using one or more detection reagents, such as, for example a second antibody that recognizes the anti-sFas antibody. The second antibody is then detected and reflects the amount of sFas present in the sample.

In a further specific, but non-limiting implementation, the method includes contacting the sample obtained from a subject with a sFas binding agent and one or more detection reagents which indicate the level of sFas in the sample.

The detection reagent useful in the method and the kit defined herein reflects the amount of sFas in a sample. A detection reagent may be any suitable compound that allows for the measurement of sFas in an assay. A suitable detection reagent is for example, a secondary antibody. Other possible detection agent may include other types of molecules, such as, proteins, polypeptides, carbohydrates, nucleic acids, lipids, and the like. The method and kits defined herein may use more than one detection reagents.

The detection reagent may be labeled by a detection label. A suitable detection label may be an enzyme, a fluorescent label, radioactivity, a colour compound which may be measured by spectrophotometric or fluorometric methods, or a compound that emits, absorbs or converts energy. The detection labeled may be a detachable label that is attached, by any suitable means, to the detection agent.

In another specific, but non-limiting, implementation, the method for diagnosing ACS in a subject includes attaching or affixing a sFas binding agent to a substrate or a support and contacting the substrate or support with a sample obtained from the subject. sFas that may be present in the sample interacts with the sFas binding agent on the substrate or support to form a complex that is detectable and measurable. The method may also include exposing the complex to one or more detection reagents. The detection reagents generate a detectable signal which indicates the amount or concentration of sFas in the sample.

In a specific implementation, the method includes comparing the amount, levels or concentration of sFas in the sample to a reference value. Comparison of sFas level with the reference value indicates the presence or absence of ACS.

The term “immunoassay” as used herein refers to an analytical method which uses the ability of an antibody to bind a particular antigen as the means for determining the presence of the antigen or antibody. It is contemplated that a range of immunoassay formats be encompassed by this definition, including but not limited to direct immunoassays, indirect immunoassays, and “sandwich” immunoassays. A particularly preferred format is a sandwich enzyme-linked immunosorbent assay (ELISA). However, it is not intended that the technology defined herein be limited to this format. It is contemplated that other formats, including radioimmunoassays (RIA), immunofluorescent assays (IFA), and other assay formats, including, but not limited to, variations on the ELISA method will be useful in the method of the present technology. Thus, other antigen-antibody reaction formats may be used in the present technology, including but not limited to “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen); “complement fixation” (i.e., the use of complement in an antibody-antigen reaction method), and other methods commonly used in serology, immunology, immunocytochemistry, histochemistry, and related fields. The techniques for carrying out an immunoassay method are well known in the art.

Enzyme-Linked ImmunoSorbent Assay, also called ELISA, Enzyme immunoassay or EIA, is a biochemical technique used to detect the presence of an antibody or an antigen in a sample. In particular, an ELISA assay be used to measure the level of sFas in a sample.

The ELISA has been used as a diagnostic tool in medicine and plant pathology, as well as a quality control check in various industries. In simple terms, in ELISA an unknown amount of antigen is affixed to a support, and then a specific antibody is washed over the surface so that it can bind to the antigen. This antibody is linked to an enzyme, and in the final step a substance is added that the enzyme can convert to some detectable signal. Thus in the case of fluorescence ELISA, when light of the appropriate wavelength is shone upon the sample, any antigen/antibody complexes will fluoresce so that the amount of antigen in the sample can be inferred through the magnitude of the fluorescence.

In a particular implementation, an immunoassay of the method of the present technology can be performed by contacting a sample that is susceptible of containing sFas (antigen), under conditions sufficient for binding of sFas to anti-sFas antibodies. sFas is quantified by detecting complex(es) comprising sFas bound to the anti-sFas antibodies. Such assay may be performed by affixing the sFas antigen or the anti-sFas antibodies to a support.

Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA, defined below). After the antigen is immobilized, the detection antibody is added, forming a complex with the antigen. The detection antibody can be covalently linked to an enzyme, or can itself be detected by a secondary antibody which is linked to an enzyme through bioconjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample.

Generally, in a sandwich ELISA, a support is coated with a capture antibody, a sample is added and any antigen present in the sample binds to the capture antibody. A detecting antibody (enzyme-linked secondary antibody) is added and binds to a second epitope on the antigen forming an antibody: antigen: antibody complex. A substrate is added and is converted by the enzyme linked to the secondary antibody thereby producing a colored product or emitting light that is proportional to the concentration of the antigen bound. As a variant, a chemoluminescent sandwich ELISA may be used in combination with a luminometer or a color sandwich ELISA using alkaline phosphatase-based color amplification system that is read at a specific wavelength. It will be appreciated that more then one type of secondary antibodies may be used in this technique. In a specific, but non-limiting example, the concentration of capture antibody used to coat the support is typically between 20 μg to 40 μg per 200 μl buffer per well of a microliter plate. These techniques are well known in the art.

For example, in one implementation of he present technology, the anti-sFas antibodies are affixed to a support, any sFas present in the sample binds to antibodies affixed to the support, thereby forming a support-affixed complex. A detection reagent, which may be labeled (direct or indirect label, such as, for example, a secondary labeled anti-sFas antibody, is added and binds to sFas captured by the first antibody. The bound entities are separated, if necessary, from free labeled secondary antibody, typically by washing, and the signal from the bound label is detected.

Detection of an antibody-antigen complex can be performed by several methods. The mobile antigen or antibody or detection reagent may be prepared with a label such as biotin, an enzyme, a fluorescent marker, or radioactivity, and may be detected directly using this label. Appropriate reporter reagents may then be added to detect the labeled component. Any number of additional antibodies may be added as desired. These antibodies may also be labeled with a label, including, but not limited to an enzyme, fluorescent marker, or radioactivity. Either the antigen or the antibody (primary or secondary) may be immobilized on a support, but the labeled component cannot be immobilized because the detectable signal is precluded from being a measure of binding.

In a specific, but non-limiting implementation, sFas levels in a sample are measured by affixing anti-sFas antibodies to a support such as, for example, onto microwells. Any sFas present in a sample binds to anti-sFas antibodies affixed to the support. A biotin-conjugated anti-sFas antibody is added and binds to sFas captured by the first antibody. Following incubation, unbound biotin-conjugated anti-sFas antibody is removed during a wash step.

Streptavidin-HRP is added and binds to the biotin-conjugated anti-sFas antibody. Following incubation, unbound Streptavidin-HRP is removed during a wash step, and substrate solution reactive with HRP is added to the support. A colored product is formed in proportion to the amount of sFas present in the sample. The reaction is terminated by addition of an acid and absorbance measured at 450 nm. Skilled artisans in the art will appreciate that may other variants of this specific implementation are encompassed by the present technology.

As used herein, the term “support” is used in reference to any solid material to which reagents such as antibodies, antigens, and other compounds may be attached. In some instances, the solid support may be a microliter plate. In some other instances, the solid support may be a diagnostic devide for individual use or for automated purposes. For implementations of the technology that make use of support, the support can be any .suitable material with sufficient surface affinity to bind, for example, a sFas binding agent, such as a natural polymeric carbohydrates and their synthetically modified, crosslinked, or substituted derivatives, such as agar, agarose, cross-linked alginic acid, substituted and cross-linked guar gums, cellulose esters, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali. and alkaline earth metals, aluminum and magnesium; and aluminum or silicon oxides or hydrates, such as clays, alumina, talc, kaolin, zeolite, silica gel, or glass; and mixtures or copolymers of the above classes.

The support may be used in a shape such as films, sheets, membranes, beads, tubes, particulates, plates, or they may be coated onto, affixed, attached, bonded, or laminated to appropriate carriers, such as paper, glass, plastic films, fabrics, as well as many other items which will be apparent to a person skilled in the art.

Commonly used cross-linking agents for immobilizing an antibody or an antigen to the support include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), bifunctional maleimides such as bis-N-maleimido-1,8-octane. Derivatizing agents such as methyl-3-((p-azidophenyl)-dithio) propioimidate yield photoactivatable intermediates capable of forming cross-links in the presence of light.

If 96-well plates are utilized for assaying samples, they are preferably coated with the mixture of anti-sFas antibodies typically diluted in a buffer such as 0.05 M sodium carbonate by incubation for at least about 10 hours, more preferably at least overnight, at temperatures of about 4-20° C., more preferably about 4-8° C., and at a pH of about 8-12, more preferably about 9-10, and most preferably about 9.6. If shorter coating times (1-2 hours) are desired, one can use 96-well plates with nitrocellulose filter bottoms or coat at 37° C. The plates may be stacked and coated long in advance of the assay itself, and then the assay can be carried out simultaneously on several samples in a manual, semi-automatic, or automatic fashion, such as by using robotics.

The coated plates are then typically treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin, egg albumin, casein, and non-fat milk. The blocking treatment typically takes place under conditions of ambient temperatures for about 1-4 hours, preferably about 1.5 to 3 hours.

After coating and blocking, the standard or the sample to be analyzed, appropriately diluted, is added to the immobilized phase. The preferred dilution rate is about 5-15%, preferably about 10%, by volume. Buffers that may be used for dilution for this purpose include (a) phosphate-buffered saline (PBS) containing 0.5% BSA, 0.05% TWEEN20 detergent (P20), 0.05% PROCLIN 300 antibiotic, 5 mM EDTA, 0.25% 3-((3-cholamidopropyl)dimethylammonio)-1-propanesulphonate (CHAPS) surfactant, 0.2% beta-gamma globulin, and 0.35M NaCl; (b) PBS containing 0.5% bovine serum albumin (BSA), 0.05% P20, and 0.05% PROCLIN 300, pH 7; (c) PBS containing 0.5% BSA, 0.05% P20, 0.05% PROCLIN 300, 5 mM EDTA, and 0.35 M NaCl, pH 6.35; (d) PBS containing 0.5% BSA, 0.05% P20, 0.05% PROCLIN 300, 5 mM EDTA, 0.2% beta-gamma globulin, and 0.35 M NaCl; and (e) PBS containing 0.5% BSA, 0.05% P20, 0.05% PROCLIN 300, 5 mM EDTA, 0.25% CHAPS, and 0.35 M NaCl. PROCLIN 300 acts as a preservative, and TWEEN20 acts as a detergent to eliminate non-specific binding. The added EDTA and salt of buffer (a) act to decrease the background over the other buffers, including buffer (b).

In a specific implementation, for sufficient sensitivity, the amount of sFas-containing sample added may be such that the immobilized anti-sFas antibodies are in molar excess of the maximum molar concentration of sFas anticipated in the sample after appropriate dilution of the sample. This anticipated level depends mainly on any known correlation between the concentration levels of the free sFas in the particular sample being analyzed with the clinical condition of the patient. Thus, for example, an adult patient may have a maximum expected concentration of sFas in the sample (e.g. plasma) that is quite high, whereas a child will be expected to have a lower level.

The detection labels suitable for use in the present technology include any composition detectable by spectroscopy, photochemical, biochemical, immunochemical, electrical, optical, or chemical means. Useful labels in the present invention include magnetic beads, chemiluminescent compounds, fluorescent dyes such as a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-natpthalenesulfonyl chloride (DANSC), tetramethylrhodamine isothiocyanate (TRITC), lissamine, rhodamine 8200 sulphonyl chloride (RB 200 SC), and the like.

Radioactive elements are also useful detection labels. An exemplary radiolabeling agent is a radioactive element that produces gamma ray emissions. Elements which themselves emit gamma rays, such as ₁₂₄I, ¹²⁵I , ¹²⁸I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. Also useful is a beta emitter, such as ¹¹¹indium or ³H.

The linking of labels, i.e. labeling of peptides and proteins, is well known in the art, e.g. by protein conjugation or coupling through activated functional groups, and will be recognized by skilled artisans.

Imunoassays according to the present technology may also be carried out using techniques such as electrochemical detection systems fluorescent polarization, scanning probe microscopy, micro electromechanical systems. A person skilled in the art pertaining to the present technology will be familiar with these techniques and the steps to practice them.

In a further specific, but-non limiting, implementation, of the present technology, multiple analytes or markers in a single sample may be measured simultaneously by for example, affixing a plurality of binding agents on the support, including one that is specific for sFas. Each binding agent on the support is intended to test the presence of different analytes or markers in the sample. Such method may also involve the use of different detection reagents which indicate the presence and the amount of a particular analyte or marker in the sample.

Taking any necessary dilution of the sFas-containing sample into account, the final concentration of the sFas will normally be determined empirically to maximize the sensitivity of the assay over the range of interest. As a general guideline, the molar excess is suitably less than about ten-fold of the maximum expected molar concentration of autoantibody in the sample after any appropriate dilution of the sample.

Other possible methods for measuring the levels of sFas in a sample include, but are not limited to, methods wherein the amount of sFas is measured by assessment of its biological activity or biological function.

Such methods include competitive-binding assays in which a biologically specific binding agent competes for radioactively labeled or unlabeled compounds. In a specific example, the amount of sFas in a sample may be measured by contacting the sample with a radioactively labeled Fas-L. By measuring the amount of Fas-L bound to sFas, one can then determine the amount of sFas in the sample.

A person skilled in the art will appreciate that such method may also be performed using a biological substrate of Fas, such as for example, downstream members of the Fas signaling pathway, for example, Fas-associated proteins or other proteins with a death effector domain (DED).

In a further implementation, sFas can also be assessed by dot blot detection assays, using standard methodologies and apparatus.

In a further specific, but non-limiting, implementation, a diagnosis method for the presence of ACS in a subject is performed by determining the level of Fas mRNA in a sample. Methods for measuring the level of mRNA include, but are not limited to, Northern blot assays. Other, methods for measuring Fas mRNA are well known in the art.

Other possible methods for measuring the levels of sFas in a sample include, but are not limited to methods wherein the amount of sFas is measured by assessment of its biological activity or biological function or through measurement of the Fas-encoding gene expression.

In a further specific implementation, sFas or fragment thereof or sFas:sFas binding agent complex as defined above may be detected based on further characteristics. The characteristics that can be measured in the present technology include, but are not limited to, retention time, molecular weight, buoyant density, fluorescence polarization, poly-ethylene glycol (PEG) precipitation, and/or those known in the art.

The present technology is not limited to the assay methods defined above. As will be appreciated by a person skilled in the art, any suitable detection method that allows for the specific measurement of sFas or fragment thereof may be utilized.

The methods defined herein are practiced in accordance with standard practice and may include the use of positive and/or negative controls and/or or standards containing known concentrations of sFas.

In a specific, but not limiting implementation, the methods defined herein include comparing the amount of sFas in a sample to a reference value. As used herein, the expression “reference value” refers to a value which dictates if the subject's condition is pathological or if the subject's condition is normal. In a specific, but non-limiting implementation, comparison of the amount of sFas in a sample with a reference value indicates the absence or the presence of ACS. Any increase in the test sample relative to the reference value can be assessed for significance by conventional statistical methods.

In a specific, but non-limiting, implementation, an amount of sFas that is equal to or that exceeds the reference value indicates the presence of ACS, whereas an amount of sFas that is below the reference value indicates the absence of ACS.

In another specific implementation, the method also includes comparing the amount of additional ACS markers (such as, for example, traditional ACS markers or necrosis factors) in a sample with reference values. The reference values indicate the absence or the presence of ACS. However, as indicated above, the indication provided by traditional ACS markers is likely not to permit an earlier diagnosis of ACS than the indication provided by sFas.

A person skilled in the art will appreciate that the reference value to which is compared the level of sFas may differ from the reference value to which is compared the level of the additional ACS markers, and that these reference values may be obtained under different clinical conditions.

The reference value may take the form of a range of, values, a cut-off number, a curve, a plot, a graph, to name a few, which establish the likelihood that the subject suffers from ACS.

In some aspects, the reference value may be affected by one or more clinical variables such as, but not limited to, a history of congestive heart failure, renal function, electrocardiographic changes, a history of coronary artery disease, body mass index and diabetes.

The reference value is preferably based on results obtained from diagnostic tests performed on a large pool of subjects which may include the normal or healthy subjects as controls.

In a specific, but non-limiting, implementation, the amount of sFas in a sample obtained from a subject is compared to a reference value which is extrapolated from measurements of sFas in different subjects (i.e., not including the subject being diagnosed), more preferably subjects with ACS. In an alternate implementation, the amount of sFas in a sample obtained from a subject is compared to a reference value which is extrapolated from measurements of sFas in the subject himself.

Measurements of sFas levels in a sample obtained from a subject may be obtained over a period of time and may be used to assess progression or regression of sFas expression in the subject over time.

In a specific, but non-limiting, implementation, the reference value is established using the receiver operating characteristic curves (ROC). As used herein, ROC refers to a plot of the sensitivity (or the true positive value) versus the false positive. This analysis allows for optimization of the results obtained from the test and the clinical outcome.

In one specific, but non-limiting, implementation, the reference value may be obtained by measuring the concentration of sFas in a sample, wherein a positive result, i.e. the presence of ACS, is indicated by a concentration equal or greater than the reference value. In this implementation, the reference value is thus a cut-off value and refers to the sample's concentration of sFas.

In another specific, but non-limiting, implementation, the reference value may be obtained by measuring the concentration of sFas in a sample and transforming the concentration to its natural logarithm, wherein a positive result, i.e. the presence of ACS, is indicated by a natural logarithm-transformed value equal or greater than the reference value. In this implementation, the reference value is thus a cut-off value and refers to the natural logarithm in pg per mL of sample. For example, the cut off value is 3.68, preferably 4.90, most preferably 5.06, calculated as the natural logarithm in pg per mL of sample.

A person skilled in the art will appreciate that the reference value may differ from one user of the methods and the kits defined herein to another, depending on various external and internal factors such as, but not limited to, laboratory conditions, materials and reagents used for the assay methods, quality of the sample, management of the sample, and the like, which will be apparent to a person skilled in the art.

Kits and Packages for the Diagnosis of ACS

The present technology also provides kits for the diagnosis of a subject with ACS. The kits defined herein are useful for routine clinical use for diagnosing ACS. In a specific implementation, the kit is a clinical kit. The kit may allow for the diagnosis of one or more subjects simultaneously.

It is desirable that the kit rapidly provides results. More specifically, the kit should provide results within about .2 hours, more preferably within about 1 hour and most preferably within about 30 minutes. It is also desirable that the kit be adapted to directly process a sample obtained from a subject.

The kit may determine the amount of sFas using the assays and methods defined herein. For example, by utilizing pre-packaged diagnostic kits comprising at least a sFas binding agent (for detection of sFas or fragments or derivatives thereof) which can be conveniently used, e.g., in clinical settings to diagnose ACS.

The kit may comprise components useful in an ELISA method for detecting sFas in a sample. In a specific, but non-limiting, implementation, the kit may also be based on a ELISA, which technique is well known in the art.

The kit may further comprise a support, such as a matrix support or a clinical diagnosis support, and a sFas binding agent, for example an anti-sFas antibody, which may be immobilized on the support such as being coated on a microtiter plate. The sFas binding agent may also be immobilized on any other type of support which allows convenient and rapid clinical assessment of sFas levels in a sample, such as, for example, a dipstick.

The kit may further comprise one or more detection reagents, such as defined above, which indicate the amount of sFas present in the sample. The kit may further comprise purified reagents, such as a purified antibody of interest, as a standard.

As used herein, the term “packaged” can refer to the use of a solid matrix or material such as glass, plastic, paper, fiber, foil and the like capable of holding within fixed limits the reagents of the kit.

Thus, for example, a package or a kit can be a glass vial used to contain milligram quantities of a contemplated sFas binding agent or it can be a microtiter plate well to which microgram quantities of a contemplated sFas binding agent has been operatively immobilized.

Alternatively, a package or kit could include sFas binding agent-coated microparticles entrapped within a porous membrane or embedded in a test strip or dipstick, etc.

Alternatively, the sFas binding agent can be directly coated onto a membrane, test strip or dipstick, etc. which contacts the sample. Many other possibilities exist and will be readily recognized by those skilled in this art.

Instructions for use typically include a tangible expression describing the reagent concentration or at least one assay method parameter such as the relative amounts of reagent and sample to be admixed, maintenance time periods for reagent/sample admixtures, temperature, buffer conditions and the like.

The kit of the present technology may further include a detection label or indicating means capable of signaling the formation of a complex between for example, sFas and a sFas binding agent.

As a specific, but non-limiting example, the kit comprises components for detecting and/or measuring sFas. Such components include a sFas binding agent such as an anti-sFas antibody. Such sFas binding agent allows sFas to be captured effectively. The sFas binding agent may be linked to a support. The kit may also comprise means for detecting the sFas binding agent bound to sFas. Such means for detection may be, for example, an antibody directed toward the constant region of human IgG (e.g., rabbit anti-human IgG antibody), which may itself be detectably labeled (e.g., with a radioactive, fluorescent, calorimetric or enzyme label), or which may be detected by a labeled secondary antibody (e.g., goat anti-rabbit antibody).

The methods and kits defined herein may also include, in the same or as a separate package, one or more detection reagents, which are capable of selectively binding sFas or sFas binding agent or a complex containing either species, but which is not itself necessarily sFas or sFas binding agent. Exemplary specific detection reagents are second antibody molecules, e.g. anti-human antibodies, complement proteins or fragments thereof, S. aureus protein A, and the like. The specific detection reagents may bind the antibody or antigen when it is present'as part of a complex.

In an exemplary setup, a sFas binding agent is coated or adsorbed on to the surface of a support. A test sample from a subject is contacted with the immobilized sFas binding agent and any sFas which may be present in the sample bind to the sFas binding agent.

In another exemplary setup, the sFas binding agent may be labeled with a label. The label is any entity that is capable of being conjugated or bound to the sFas binding agent and that is capable of being detected by an analytical technique. The label may be conjugated or bound to the sFas binding agent prior to or after contacting the sFas binding agent with the sFas/sample.

Detection of the label is an indication that sFas is present in the sample. If the label cannot be detected, then this is an indication that sFas is not in the sample. Since the presence of sFas is correlated with a disease condition (specifically ACS), the presence or absence of sFas is an indication of whether the subject has the condition being tested or not.

Using the anti-sFas antibody as an example of a sFas binding agent, the label may be a chemical moiety capable of being detected by an analytical technique, the chemical moiety being conjugated to the anti-sFas antibody. In this embodiment, the chemical moiety is generally conjugated to the anti-sFas antibody before the anti-sFas antibody is contacted with the sFas/sample.

When the method or kit includes a sFas binding agent that is not labeled, one or more detection reagents are typically used to indicate the amount of sFas. The one or more detection reagents may themselves be labeled. The label may be another antibody or a collection of other antibodies having conjugated thereto a chemical moiety that is capable of being detected by an analytical technique. Amplifying means or reagents may also be included to amplify the signal of the label.

The reagents of any diagnostic system or kit defined herein can be provided in solution, as a liquid dispersion or as a substantially dry powder, e.g., in lyophilized form. A support such as the above-described microtiter plate or diagnostic device, and one or more buffers can also be included as separately packaged elements in the diagnostic assay systems or kits of this technology.

The system or kit may further comprise a washing solution. Washing is preferably accomplished using a washing solution comprising a buffer, such as phosphate buffered saline solution. An emulsifier may also be present in the washing solution.

The kit is not limited to the detection methods outlined above. A person skilled in the art will appreciate that other techniques useful for determining the quantity of sFas in a sample may be useful in a kit for diagnosing ACS. Such other techniques may include determining the quantity of sFas through functional assays such as defined above.

In a further specific, but non-limiting, implementation of the present technology, the methods and kits defined herein are non-invasive.

Experiments and Data Analysis

A cohort used for the present study had a very high probability of ACS (80% prevalence): by definition, to be recruited in this study, patients were initially believed to suffer from ACS. In this patient population, measuring sFas could have helped in improving diagnosis accuracy. For instance, values greater than 5.06 were associated with a 95% positive predictive value PPV for ACS.

Because sFas increases rapidly in ACS, this leads to earlier diagnosis and management in patients with atypical presentations. This cut-off value also had excellent negative predictive value (NPV) (98%). Using the lower cut-off point of 4.9, the NPV was 100%, with a 95% CI of 99-1000. Discharging patients with lower values would have resulted in less than 1% erroneous early discharge in patients who would have had a final diagnosis of ACS, while avoiding unnecessary observation in 77% of patients who have non-cardiac chest pain.

Increased levels of sFas can originate from various cells undergoing apoptosis, including VSMC, macrophages and myocardial cells. Hence, its potential clinical use is to enhance diagnostic accuracy in atypical cases and to hasten aggressive management or early discharge. The latter is particularly pertinent since the majority of patients in emergency departments with chest pain turn out not to have ACS^{22;23}.

The data presented herein show that sFas, a plasma marker of apoptosis, is increased in patients with ACS. It improves diagnostic accuracy over models that include risk factors for coronary artery disease and case-definition variables. It has excellent NPV, even in a cohort with high pre-test probability. Since sFas is rising rapidly after the onset of myocardial ischemic symptoms, sFas is a reliable biomarker for early diagnosis or discharge in the frequently atypical chest pain.

### EXAMPLE 1

Patients and Study Design

The present study is a cross-sectional study, which is nested within a prospective cohort, the RISCA (acronym for Récurrence et inflammation dans les syndromes coronariens aigus) study. The recruitment phase of the RISCA study began in 2000 and ended in early 2002. In all, 1210 patients participated in this study. The number of participating centers (n=8) was deliberately limited to a small and balanced mix of secondary and tertiary centers to fully reflect the spectrum of practice patterns. All patients hospitalized with an initial admission diagnosis of an ACS (UA or MI) were eligible for the RISCA study if they were approached within 24 hours of the end of ischemic symptoms. Clinical management was left to the discretion of treating physicians. For inclusion in this cohort, the initial diagnosis of UA required either one episode of typical chest pain at rest or with minimal exertion lasting ≧10 min or ≧2 episodes each lasting min. This could be either new-onset angina or an abrupt and significant change in the pattern of established angina, with CK-MB remaining in the normal range (<1.5 times the upper normal limit). For inclusion purposes, an MI episode was defined as characteristic discomfort or pain with an elevation of CK-MB to ≧1.5 times the upper normal limit, or CK≧2 times the upper normal limit if no CK-MB were measured, or with an ECG showing a greater than 1 mm ST-elevation in 2 contiguous leads or pseudonormalization.

Among the 1210 patients initially recruited on the basis of the latter criteria, 100 were eventually discharged with a final diagnosis of non-cardiac chest pain or chest pain of undetermined etiology (no ACS group). sFas and traditional markers were measured in all these patients, as well as in a third of patients who did have final diagnosis of MI or UA at discharge (ACS group). The latter patients were selected for another study that was performed concurrently. All patients who had recurrent ACS or cardiovascular death at 1 year and their randomly selected sex- and age-matched controls formed the ACS group in the present study.

Data collection

Experimental blood samples were taken within 24 hours of the end of chest pain. After centrifugation, plasma samples were distributed in aliquots of 2 ml each, stored locally at −70° C. and sent on dry ice to the central repository where they were stored at −80° C. Clinical data collection was performed prospectively by trained research nurses at each study site and systematically validated by chart review by the central organizers.

### EXAMPLE 2

Measurement

Predictive Markers

Citrated plasma levels of sFas were measured at baseline using commercially available enzyme-linked immunosorbent assay (ELISA) kit (Bender MedSystems, Vienna, Austria). The coefficient of variability. among 33 plaques was 6.33%.

Covariates

Detailed clinical variables as well as glomerular filtration rate (GFR-MDRD abbreviated equation¹¹), maximal CK, CK-MB, cardiac troponin T, and high-sensitivity CRP were recorded so as to permit the independent, incremental value of sFas. All measurements for CRP and troponin were performed in a single batch. CRP was measured with the N High-Sensitivity CRP mono assay using the BN ProsPec Nephelometer (Dade Behring, Deerfield, Ill.). In addition to each centre measuring cardiac troponin T or I for clinical purposes, cardiac troponin T was also measured centrally using a commercial assay (Roche Inc., Mannheim, Germany) with a detection limit for myocardial injury of 0.1 μg/L.

Baseline ECG and Left Ventricular Function

The admission ECG was centrally evaluated in all patients. Ischemic changes were defined as: ≧1 mm ST segment elevation or depression, or ≧1 mm T wave inversion in at least 2 contiguous leads. Baseline left ventricular ejection fraction (LEVF) was measured by echocardiography, scintigraphy or angiography.

Outcome

The main outcome was the relationship between a discharge diagnosis of ACS (MI or UA) and soluble apoptosis markers. The criteria for a discharge diagnosis of MI were: characteristic discomfort or pain with an elevation of CK-MB to times the upper normal limit. If no CK-MBs were performed, total CK had to be 2 times the upper normal limit with raised troponin (according to the cut-off defined by each site's laboratory). The final discharge diagnosis of UA was made by the treating physicians and recorded from the medical chart.

Statistical Analyses

Normally distributed variables are presented as mean and standard deviation (SD), and non-normally distributed variables, as median with interquartile range (25th and 75th percentile). Categorical variables are summarized using proportions. Natural log transformation of sFas was performed since these markers were not normally distributed. Initial bivariate analyses were performed to identify potential confounders. Spearman correlation coefficients (continuous covariates) were also used to assess the relationship between the markers and covariates. Associations between sFas level and the diagnosis of ACS were assessed using logistic regression, in univariate and multivariate models. Centres were included as independent variables in initial multivariate models. The incremental value of sFas measurement was studied by comparing the area under the receiver operating curve (ROC), or C statistic¹², for models with and without sFas measurement, in the presence of all standard diagnostic criteria. Finally, to compare temporal trends in markers in patients with an ACS, simple linear regression models were used in which log transformed sFas or troponin were included as dependent variables and time since the beginning of symptoms as the independent variable.

### EXAMPLE 3

Results

sFas was measured in 488 patients, 223 with a diagnosis of MI, 165 with a diagnosis of UA and 100 with a final discharge diagnosis of non-cardiac chest pain. Patients who did not have a discharge diagnosis of ACS were more likely to be younger, female, and to have higher creatinine clearance and left ventricular ejection fraction (LVEF), and less likely to have previous Congestive heart failure (CHF) or insulin-dependent diabetes (see Table 1). By definition, their CK-MBs and troponin were not elevated, and only 21% had ischemic ECG changes as described above. sFas levels were higher in the ACS group.

TABLE 1
Baseline characteristics and markers of apoptosis
according to outcome status (n = 488)

| | No ACS | | MI | | UA | |
|---|---|---|---|---|---|---|
| Baseline characteristics | (n = 100) | | (n = 223) | | (n = 165) | |
| Age in years (±standard | 62 | (10) | 64 | (12) | 67 | (11) |
| deviation (SD)) | | | | | | |
| Gender-male (%) | 61 | (61) | 166 | (74) | 107 | (65) |
| Previous coronary artery | 56 | (56) | 70 | (31) | 110 | (67) |
| disease (%) | | | | | | |
| Previous vascular disease | 26 | (26) | 38 | (17) | 57 | (35) |
| (%) | | | | | | |
| Previous congestive heart | 2 | (2) | 18 | (8) | 20 | (12) |
| failure (%) | | | | | | |
| Hypertension (%) | 58 | (58) | 103 | (46) | 117 | (71) |
| Smoking status (%) | | | | | | |
| Active smoker | 26 | (26) | 70 | (31) | 35 | (21) |
| Past smoker | 59 | (59) | 104 | (47) | 93 | (56) |
| Never smoked | 15 | (15) | 49 | (22) | 37 | (22) |
| Dyslipidemia (%) | 72 | (72) | 116 | (52) | 165 | (82) |
| Diabetes (%) | 22 | (22) | 47 | (21) | 40 | (24) |
| Insulin-dependent (%) | 0 | (0) | 17 | (8) | 13 | (8) |
| Left ventricular ejection | 60 | (55-65) | 52 | (45-60) | 60 | (48-65) |
| fraction (%)- | | | | | | |
| Median and Inter-Quartile | | | | | | |
| Range (IQR) | | | | | | |
| Body mass index in kg/m² | 27 | (4) | 27 | (4) | 27 | (4) |
| (±SD) | | | | | | |
| Creatinine clearance- | 71 | (18) | 66 | (17) | 63 | (19) |
| m1/min/1.73 m² (±SD) | | | | | | |
| Maximal CK (IQR) | 82 | (57-119) | 762 | (269-3653) | 87 | (61-150) |
| Ratio CK-MB/upper normal | 0 | (0) | 223 | (100) | 0 | (0) |
| limit ≧1.5 (%) | | | | | | |
| Troponin (IQR) | 0.01 | (0.01-0.01) | 1.39 | (0.41-3.31) | 0.01 | (0.01-0.09) |
| Baseline ECG with ischemic | 21 | (21) | 159 | (71) | 62 | (38) |
| changes (%) | | | | | | |
| Baseline sFas in pg/ml | 3.15 | (2.46) | 5.95 | (0.46) | 5.86 | (0.34) |
| (±SD)* | | | | | | |

Legend:
ACS: acute coronary syndromes
MI: myocardial infarction
UA: unstable angina
*Reported values are transformed in the natural log

Higher sFas levels were weakly associated with increased age (r=0.13, 95% confidence interval (CI) (0.04, 0.22)), lower glomerular filtration rate GFR (r=−0.09, 95% CI (−0.17, 0.00)) and higher baseline C-reactive protein (CRP) levels (r=0.11, 95% CI (0.03, 0.20)). The correlation between sFas and troponin (r=0.34, 95% CI (0.26, 0.42)) and maximal CK (r=0.22, 95% CI (0.13, 0.30)) was somewhat stronger. Patients with a history of CHF also showed higher levels. There was no confounding by centre in the relationship between sFas and ACS. However, centres were not included in the final multivariate analyses for sake of model parcimony in the absence of a significant change in the clinical interpretation of the results.

Higher sFas levels, CRP, age, previous CHF, as well as lower GFR and LVEF were associated with a final diagnosis of ACS in univariate analyses (see Table 2). Two multivariate models are reported. The first includes all potential confounders identified on bivariate analyses, but not the case definition biomarker variables CK and troponin or ischemic ECG changes. The second also includes the case definition variables, to evaluate whether sFas measurement had incremental value in the diagnosis of ACS. sFas remained a strong predictor of having a discharge diagnosis of ACS after adjusting for confounders and case definition variables (OR: 7.59 for a natural log unit change, 95% CI (3.86-14.92)).

TABLE 2
Multivariate regression models for the diagnosis
of ACS (n = 488) with and without case definition variable

| | | Adjusted OR | Adjusted OR |
|---|---|---|---|
| | | (95% CI) | (95% CI) |
| | | Without case | With case |
| Baseline | Crude OR | def. | def. |
| characteristics | (95% CI) | variables* | variables** |
| Baseline sFas | 11.29 (6.36-20.03) | 10.56 (5.76-19.37) | 7.59 (3.86-14.92) |
| (per 1 ln unit) | | | |
| Age (per year) | 1.03 (1.01-1.05) | 0.99 (0.95-1.03) | 0.99 (0.94-1.04) |
| Gender-female | 0.67 (0.42-1.05) | 0.56 (0.24-1.26) | 0.87 (0.32-2.38) |
| Previous | 0.68 (0.44-1.06) | 0.77 (0.33-1.80) | 2.20 (0.81-5.98) |
| coronary | | | |
| artery | | | |
| disease | | | |
| Previous | 0.92 (0.56-1.52) | 0.58 (0.26-1.32) | 1.20 (0.47-3.05) |
| vascular | | | |
| disease | | | |
| Previous | 5.31 (1.26-22.38) | 3.59 (0.40-32.52) | 6.10 (0.66-56.50) |
| heart failure | | | |
| Hypertension | 0.95 (0.61-1.49) | 0.90 (0.40-2.02) | 1.81 (0.71-4.59) |
| Smoking status | | | |
| Active | 1.07 (0.64-1.74) | 1.04 (0.43-2.50) | 1.57 (0.58-4.20) |
| Past or | 1.00 | 1.00 | 1.00 |
| never | | | |
| smoked | | | |
| Dyslipidemia | 0.72 (0.44-1.16) | 1.40 (0.60-3.28) | 3.16 (1.09-9.17) |
| Diabetes | 1.02 (0.60-1.74) | 1.53 (0.56-4.13) | 1.12 (0.34-3.72) |
| Ejection | 0.96 (0.94-0.98) | 1.00 (0.97-1.03) | 1.03 (0.99-1.07) |
| fraction (per %) | | | |
| Body Mass | 0.95 (0.91-0.99) | 0.95 (0.88-1.03) | 1.00 (0.90-1.11) |
| Index | | | |
| (per unit) | | | |
| Creatinine | 0.98 (0.97-0.99) | 0.98 (0.96-1.01) | 0.99 (0.96-1.02) |
| clearance | | | |
| (per ml/min) | | | |
| ln CRP (per unit) | 1.24 (1.06-1.45) | 1.20 (0.89-1.60) | 0.83 (0.56-1.22) |

*The first multivariate model includes: age, gender, previous heart failure, coronary artery disease and peripheral vascular disease, hypertension, dyslipidemia, smoking, diabetes, renal function, body mass index and CRP.
**The second multivariate model includes all of the above, plus ischemic ECG changes, troponin and diagnostic maximum CK.

Ischemic baseline ECG changes, CK and troponin were not studied on univariate analyses since they are part of case definition.

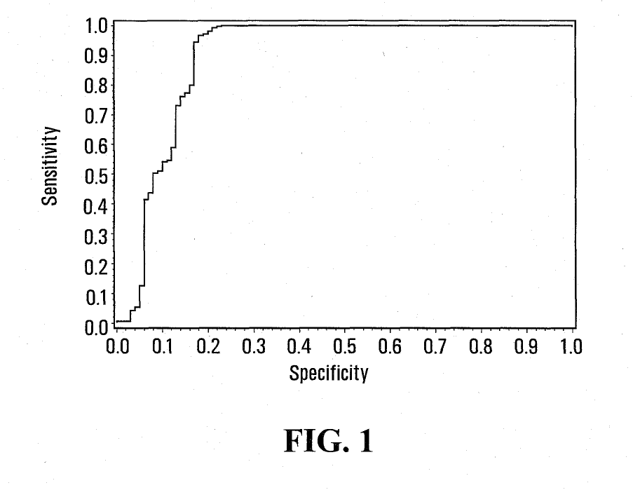

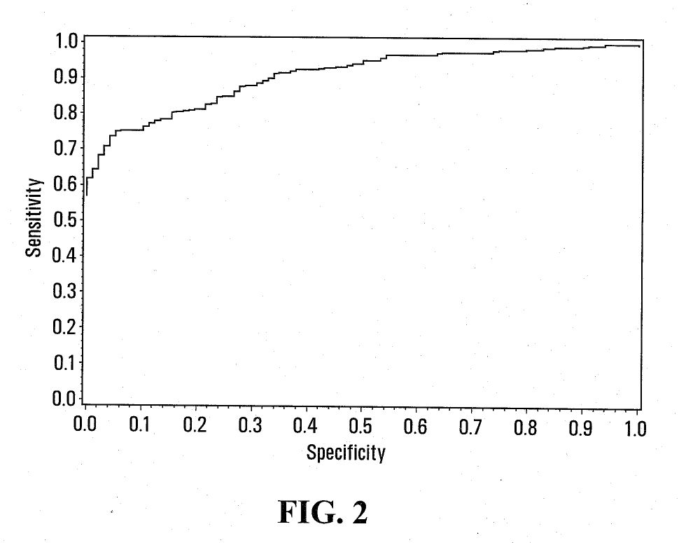

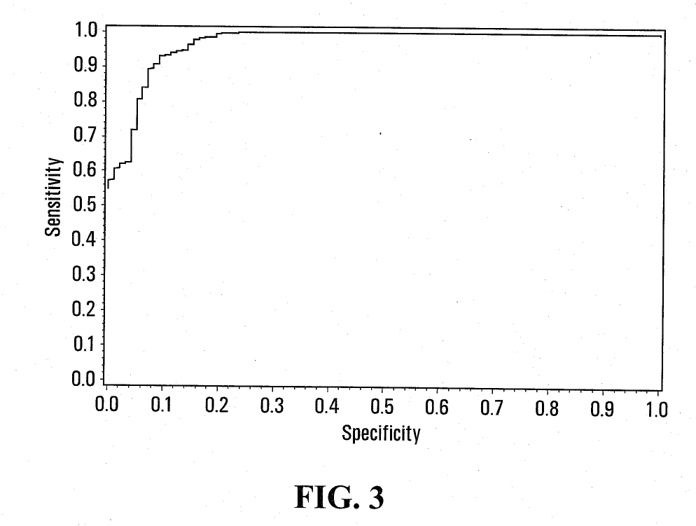

When sFas alone was used to predict diagnosis, the C statistic was 0.89, 95% CI (0.85-0.96). sFas measurement substantially improved the C statistic 0.97, 95% CI (0.96-0.99) with sFas versus 0.92, 95% CI (0.90-0.94) without sFas. The ROC curves for sFas alone, multivariate model with and without sFas are shown in FIGS. 1, 2 and 3 respectively.

In Table 3, the sensitivities, specificities, positive and negative predictive values of various diagnostic cut-off points for sFas are reported.

TABLE 3
Properties of sFas as a diagnostic test for ACS

| | | | Positive | Negative |
|---|---|---|---|---|
| Cut-off | | | predictive | predictive |
| points* | Sensitivity | Specificity | value | value |
| (percentile) | (95% CI) | (95% CI) | (95% CI) | (95% CI) |
| 3.68 | 1 (0.99-1) | 0.48 (0.44-0.52) | 0.88 (0.85-0.91) | 1 (0.99-1) |
| (10^{th} | | | | |
| percentile) | | | | |
| 5.06 | 0.994 (0.99-1) | 0.78 (0.74-0.82) | 0.95 (0.93-0.97) | 0.98 (0.97-0.99) |
| (suggested | | | | |
| cut-off) | | | | |
| 5.47 | 0.90 (0.87-0.93) | 0.83 (0.80-0.86) | 0.95 (0.93-0.97) | 0.69 (0.65-0.73) |
| (25^{th} | | | | |
| percentile) | | | | |
| 5.79 | 0.59 (0.55-0.63) | 0.87 (0.84-0.90) | 0.95 (0.93-0.97) | 0.35 (0.31-0.39) |
| (Median) | | | | |
| 6.07 | 0.32 (0.28-0.36) | 0.94 (0.92-0.96) | 0.95 (0.93-0.97) | 0.26 (0.22-0.3) |
| (75^{th} | | | | |
| percentile) | | | | |
| 6.36 | 0.11 (0.08-0.14) | 0.95 (0.93-0.97) | 0.90 (0.87-0.93) | 0.22 (0.18-0.26) |
| (90^{th} | | | | |
| percentile) | | | | |

*Values are reported in natural log of pg/ml

A sub-group analysis was performed in patients whose maximal levels of troponin and CK were in the non-diagnostic range (troponin lower than individual centre cut-off point for myocardial ischemia and CK< 2 times or CK-MB <1.5 times the upper limit of normal) and who had no ischemic changes on baseline ECG. In this sub-group, the final discharge diagnosis was UA in 69, MI in 16 and chest pain of undetermined or non-cardiac origin in 85 patients. sFas levels still showed a strong association with final discharge diagnosis of ACS on univariate (OR: 10.54 for a natural log unit change, 95% CI (4.28-25.97)) and multivariate analyses (model 1: OR: 16.67 for a natural log unit change, 95% CI (5.09-54.62)).

Median time between the initiation of symptoms and sFas measurement was 18.5 h, IQR (12.5 h-23.8 h). Cardiac troponin T levels were measured centrally on the same samples as sFas. It was compared whether troponin and sFas levels varied according to time elapsed since the beginning of symptoms in patients who had an ACS. As expected, levels of troponin increased with time since symptoms initiation (β: 0.07 per hour, 95% CI (0.04-0.10), whereas sFas did not (β: 0.00 per hour, 95% CI (−0.01-0.01), indicating a precocious increase in sFas (see Table 4).

TABLE 4
Temporal trends in the levels of sFas and cardiac
T troponin in patients with a final diagnosis of
ACS (n = 388)

| Time between | | | |
|---|---|---|---|
| beginning of | | Median sFas | Median |
| symptoms and marker | | in ln pg/ml | troponin |
| measurement | | (IQR)* | in μg/L (IQR) |
| ≦6 | hours | 5.98 (5.75-6.29) | 0.06 (0.01-0.26) |
| 6-12 | hours | 5.82 (5.61-6.08) | 0.059 (0.01-0.62) |
| 12-18 | hours | 5.87 (5.67-6.16) | 0.36 (0.02-1.79) |
| >18 | hours | 5.89 (5.68-6.10) | 0.47 (0.01-2.08) |

*Values are transformed in the natural log

All published documents mentioned in the above specification are herein incorporated by reference. Various modifications and variations of the described invention will be apparent to those skilled in the art without departing from the scope and spirit of the invention. Although the invention has been described in connection with specific preferred embodiments, it should be understood that the invention as claimed should not be unduly limited to such specific embodiments. Various modifications of the above-described modes for carrying out the invention which are obvious to those skilled in the relevant fields of technology are intended to be within the scope of the following claims.

### REFERENCES

1. Turpie A G. Burden of disease: medical and economic impact of acute coronary syndromes. Am J Manag. Care 2006; 12: S430-S434

2. Newby L K, Christenson R H, Ohman E M et al. Value of serial troponin T measures for early and late risk stratification in patients with acute coronary syndromes. The GUSTO-IIa Investigators. Circulation 1998; 98: 1853-1859

3. Fuster V, Moreno P R, Fayad Z A, Corti R, Badimon J J. Atherothrombosis and high-risk plaque: part I: evolving concepts. J Am Coll. Cardiol. 2005; 46: 937-954

4. Rossi M L, Marziliano N, Merlini P A et al. Different quantitative apoptotic traits in coronary atherosclerotic plaques from patients with stable angina pectoris and acute coronary syndromes. Circulation 2004; 110: 1767-1773

5. Myoishi M, Hao H, Minamino T et al. Increased Endoplasmic Reticulum Stress in Atherosclerotic Plaques Associated With Acute Coronary Syndrome. Circulation 2007

6. Stoneman V E, Bennett M R. Role of apoptosis in atheroscletosis and its therapeutic implications. Clin. Sci. (Lond) 2004; 107: 343-354

7. Mallat Z, Benamer H, Hugel B et al. Elevated levels of shed membrane microparticles with procoagulant potential in the peripheral circulating blood of patients with acute coronary syndromes. Circulation 2000; 101: 841-843

8. Nagata S. Fas and Fas ligand: a death factor and its receptor. Adv. Immunol. 1994; 57: 129-144

9. Fiorina P, Astorri E, Albertini R et al. Soluble antiapoptotic molecules and immune activation in chronic heart failure and unstable angina pectoris. J Clin. Immunol. 2000; 20: 101-106

10. Zhang L, Zhang L, Li Y H at al. High-dose glucose-insulin-potassium treatment reduces myocardial apoptosis in patients with acute myocardial infarction. Eur. J Clin. Invest 2005; 35: 164-170

11. Levey, A. S. Bosch J. P. Lewis J. B. et al. A simplified equation to predict glomerular filtration rate from serum creatinine. American Society of Nephrology Renal Week 2000

12. DeLong E R, DeLong D M, Clarke-Pearson D L. Comparing the areas under two or more correlated receiver operating characteristic curves: a nonparametric approach. Biometrics 1988; 44: 837-845

13. Cai W, Devaux B, Schaper W, Schaper J. The role of

Fas/APO 1 and apoptosis in the development of human atherosclerotic lesions. Atherosclerosis 1997; 131: 177-186

14. Geng Y J, Henderson L E, Levesque E B, Muszynski M, Libby P. Fas is expressed in human atherosclerotic intima and promotes apoptosis of cytokine-primed human vascular smooth muscle cells. Arterioscler. Thromb. Vasc. Biol. 1997; 17: 2200-2208

15. Schneider D B, Vassalli G, Wen S at al. Expression of Fas ligand in arteries of hypercholesterolemic rabbits accelerates atherosclerotic lesion formation. Arterioscler. Thromb. Vasc. Biol. 2000; 20: 298-308

16. Kavurma M M, Bhindi R, Lowe H C, Chesterman C, Khachigian L M. Vessel wall apoptosis and atherosclerotic plaque instability. J Thromb. Haemost. 2005; 3: 465-472

17. Martin-Ventura J L, Blanco-Colio L M, Munoz-Garcia B et al. NF-kappaB activation and Fas ligand overexpression in blood and plaques of patients with carotid atherosclerosis: potential implication in plaque instability. Stroke 2004; 35: 458-463

18. Napoli C, Quehenberger O, De Nigris F at al. Mildly oxidized low density lipoprotein activates multiple apoptotic signaling pathways in human coronary cells. FASEB J 2000; 14: 1996-2007

19. Rossi M L, Marziliano N, Merlini P A et al. Different quantitative apoptotic traits in ,coronary atherosclerotic plaques from patients with stable angina pectoris and acute coronary syndromes. Circulation 2004; 110: 1767-1773

20. Penttila I, Penttila K, Rantanen T. Laboratory diagnosis of patients with acute chest pain. Clin. Chem. Lab Med. 2000; 38: 187-197

21. Ohtsuka T, Hamada M, Sasaki O et al. Clinical implications of circulating soluble Fas and Fas ligand in patients with acute myocardial infarction. Coron. Artery Dis. 1999; 10: 221-225

22. Graff L G, Dallara J, Ross M A et al. Impact on the care of the emergency department chest pain patient from the chest pain evaluation registry (CHEPER) study. Am J Cardiol. 1997; 80: 563-568

23. Mitchell A M, Garvey J L, Kline J A. Multimarker panel to rule out acute coronary syndromes in low-risk patients. Acad. Emerg. Med. 2006; 13: 803-806

1. A method for early diagnosis of the presence of acute coronary syndromes (ACS) in a subject, comprising the steps of:
(a) determining the amount of soluble Fas (sFas) or a fragment thereof in a sample obtained from the subject; and
(b) comparing the amount of sFas or the fragment thereof in the sample with a reference value,
wherein the amount of sFas or the fragment thereof in the sample as compared with the reference value indicates the presence or absence of ACS.

2. A method for early diagnosis of the presence of acute coronary syndromes (ACS) in a subject, comprising the steps of:
(a) determining the amount of soluble Fas (sFas) or a fragment thereof in a sample obtained from the subject; and
(b) correlating the amount of sFas or the fragment thereof to the presence or absence of ACS in the subject by comparing the amount of sFas or the fragment thereof to a reference value,
wherein when the amount of sFas or the fragment thereof exceeds the reference value, ACS is diagnosed in the subject.

3. The method as defined in claim 1, wherein the amount of sFas or the fragment thereof is determined using an immunoassay or an enzyme-immunosorbent assay (ELISA).

4. The method as defined in claim 1, wherein the sample is contacted with a sFas binding agent.

5. The method as defined in claim 4, wherein the sFas binding agent is an anti-sFas antibody.

6. The method as defined in claim 4, further comprising contacting the sample with one or more detection reagents.

7. The method as defined in claim 6, wherein the one or more detection reagents is labeled.

8. The method as defined in claim 1, wherein the ACS is associated with myocardial infarction or unstable angina.

9. The method as defined in claim 1, wherein the method is non-invasive.

10. The method as defined in claim 1, wherein the method is an in vitro method.

11. The method as defined in claim 1, wherein the sample is a plasma sample.

12. The method as defined in claim 1, wherein the reference value is a cut-off value.

13. The method as defined in claim 12, wherein the cut-off value is 3.68 as calculated as the natural logarithm in pg per mL of sample.

14. The method as defined in claim 12, wherein the cut-off value is 4.90 as calculated as the natural logarithm in pg per mL of sample.

15. The method as defined in claim 12, wherein the cut-off value is 5.06 as calculated as the natural logarithm in pg per mL of sample.

16. A method for early diagnosis of the presence of acute coronary syndromes (ACS) in a subject, comprising the steps of:
(a) determining the amount of soluble Fas (sFas) or a fragment thereof in a sample obtained from a subject;
(b) determining the amount of at least one ACS marker in the sample; and
(c) correlating the amounts obtained in (a) and (b) to the presence or absence of ACS in the subject by comparing the amount obtained in (a) to a reference value for sFas and comparing the amount obtained in (b) to a reference value for the at least ACS marker,
wherein when at least the amount obtained in (a) exceeds the reference value for sFas, ACS is diagnosed in the subject.

17. The method as defined in claim 16, wherein the at least one ACS marker is selected from the group consisting of CK, CK-BM, troponin T, troponin I and myoglobin.

18. The method as defined in claim 17, wherein the at least one ACS marker is cardiac troponin T.

19. The method as defined in claim 1, wherein the subject is susceptible of having ACS.

20. The method as defined in claim 19, wherein the subject is experiencing at least atypical chest pain.

21. The method as defined in claim 20, wherein the method is performed within about 6 hours from the onset of the atypical chest pain.

22. A kit for early clinical diagnosis of the presence of acute coronary syndromes (ACS) in a subject, comprising: reagents for measuring the amount of soluble Fas (sFas) or a fragment thereof in a biological sample obtained from the subject; and instructions for use of the kit.

23. A kit for early clinical diagnosis of the presence of acute coronary syndromes (ACS) in a subject, comprising: a sFas binding agent, at least one detection reagent that indicates the amount of sFas or a fragment thereof present in a biological sample obtained from the subject; and instructions for use of the kit.

24. The kit as defined in claim 23, wherein the sFas binding agent is an anti-sFas antibody.

25. The kit as defined in claim 22 which is an enzyme-linked immunosorbent assay (ELISA) kit.

26. The kit as defined in claim 23, wherein the at least one detection reagent is suitable for use in an enzyme-linked immunosorbent assay (ELISA).

27. The kit as defined in claim 23, wherein the sFas binding agent is immobilized on a support.

28. The kit as defined in claim 23, wherein the at least one detection reagent is labeled.

29. The kit as defined in claim 28, wherein the label is adapted for reading absorbance in a colorimetric assay.

30. A kit for early clinical diagnosis of the presence of acute coronary syndromes (ACS) in a subject, comprising: a sFas binding agent, at least one sFas detection reagent that indicates the amount of sFas or a fragment thereof present in a sample, at least one ACS marker binding agent, at least one ACS marker detection reagent that indicates the amount of the ACS marker in a sample, and instructions to use the kit.

31. (canceled)
